(19) United States
(12) Patent Application Publication
(10) Pub. No.: US 2004/0176344 A1
(43) Pub. Date: Sep. 9, 2004

(54) METHOD TO TREAT PATIENTS WITH AMYOTROPHIC LATERAL SCLEROSIS AND THE LIKE

(76) Inventor: Roberta Nora Malone Rooney, North Olmsted, OH (US)

Correspondence Address: ROBERTA N. ROONEY, 6787 WARRINGTON DRIVE, NORTH OLMSTED, OH, 44070, US

(21) Appl. No.: 10/708,991

(22) Filed: Apr. 6, 2004

(51) Int. Cl. A61K 31/555; A61K 31/409

(52) U.S. Cl. 514/185; 514/410

(57) ABSTRACT

The hepatically produced isomers, URO I and URO III, are neuroprotectors capable of halting or mitigating the nervous system destruction in common neurological disorders. URO I protects neurons of the central nervous system from damage that would otherwise ensue from the neurotoxicity associated with the hepatic heme porphyrin precursors, delta-aminolevulinic acid and porphobilinogen. A method is disclosed to increase URO I to treat amyotrophic lateral sclerosis (ALS), stroke, encephalitis, meningitis, spinal cord injury and hereditary biochemical multiple sclerosis. Increases of URO III are also used to protect neurons in the peripheral nervous system in disorders including acute immunodeficiency syndrome (AIDS) related neuropathy, Guillaine-Barre syndrome and diabetic neuropathy.

### BACKGROUND OF INVENTION

1. Field of the Invention

This invention relates generally to the treatment of central nervous system (CNS) and peripheral nervous system (PNS) neuropathies, especially amyotrophic lateral sclerosis (ALS), wherein the neurotoxicity associated with the porphyrin precursors, delta-aminolevulinic acid (ALA) and porphobilinogen (PBG), is a contributing factor, and more particularly, to the use of a uroporphyrin isomer, uroporphyrin I (URO I) or uroporphyrin III (URO III) or any of their substrates as such treatment.

Normal hepatic heme synthesis is necessary to life in mammals. It is a multistage process (FIG. 1) that includes the porphyrin precursors, ALA (FIG. 4) and PBG (FIG. 5) as well as enzymes/catalysts and porphyrinogens, which are the progenitors of their respective porphyrins. The heme produced by the liver is incorporated into the hemoproteins that the liver uses to neutralize all toxins, whether they are of endogenous or exogenous origin. Levels of all of the components of hepatic heme synthesis are elevated when demand for hepatic hemoproteins to neutralize toxins is high except for the isomer I porphyrinogens, uroporphyrinogen I (UROGEN I) and coproporphyrinogen I (COPROGEN I), as they are not substrates of heme. Heme production is partially regulated by levels of production of the enzymes/catalysts, partially by the availability of iron and partially by feedback inhibition.

Hepatic Porphyrias and a Related Disorder:

The hepatic porphyrias are usually caused by inborn shortages of one or more of the enzymes/catalysts that facilitate heme synthesis (Nordmann Y; Puy H. 2002, Clin Chim Acta, 325(1-2):17-37), though some porphyrias, notably porphyria cutanea tarda (PCT) and porphyria turcica (Dean G. 1981, Arch Dermatol, (6):318), may be acquired.

The acute (neurotoxic) porphyrias cause CNS and PNS damage, elevations of porphyrins and may cause dementia. Most cause cutaneous photosensitivity as well. (Lip G Y. et al. 1993, Br J Clin Pract 47(1):38-43).

Though the precise pathogenesis of the neuron damage is unknown, elevations of the porphyrin precursors, ALA and/or PBG, are associated with the CNS and PNS destruction in several hepatic porphyrias, hereditary coproporphyria (HCP) being just one example (Kuhnel A. et al. 2000, Clin Biochem August;33(6):465-73), but most notably acute intermittent porphyria (AIP), which is the porphyria that produces the most neurological damage and exhibits the lowest production of URO I (Hindmarsh J T et al. 1999, Clin Biochem, 32(8):609-19). There are no porphyrin elevations in AIP and there is therefore no cutaneous photosensitivity. Since the enzyme/catalyst deficiency is porphobilinogen deaminase (Lundin G et al. 1997, Hum Genet 100(1):63-6) and more rarely, hydroxymethylbilane synthase (Ong P M et al. 1998, Hum Hered 48(1):24-9) there is a buildup of the two precursors, ALA and PBG, before sufficient antidotal levels of the two uroporphyrin isomers can be produced. When supplies of heme are adequate, production of the porphyrin precursors slows. This feedback inhibition mechanism is the reason that hematin is the current treatment of choice to suppress precursor production during acute attacks. Unfortunately, prophylactic use of hematin is not possible as it causes circulatory collapse (Khanderia U. 1986, Clin Pharm 5(8):690-2).

The only hepatic porphyria that is never acute (neurotoxic) is PCT which is characterized by excessive production of URO I. (Bygum A et al. 2003 Acta Derm Venereol 283(2):115-20) and also cutaneous photosensitivity. It is exacerbated by increases in iron levels and is treated by regularly scheduled phlebotomy, which reduces serum levels of iron. Menstruation makes it less severe in females of childbearing age. (Nishioka E et al. 2003, J Am Acad Dermatol, 49(3):547-50).

Another disease, currently known as hereditary biochemical multiple sclerosis (HBMS) (Rooney R. N. et al. 1999, Am J Med Genet, 86(2), 194-196) also causes motor neuron destruction indistinguishable from that seen in ALS except that neurons of the sensory cortex are also affected in HBMS. HBMS, which causes no PNS damage like the acute porphyrias do, is characterized by a shortage of URO I and an elevation in URO III (FIG. 6), indicating that URO I protects the CNS from precursor associated damage while URO III protects the PNS.

In HBMS, the shortage of URO I is caused by a lack of the iron that is needed to bridle levels of the enzymes/catalysts needed to support normal hepatic heme synthesis, uroporphyrinogen III synthase and cosynthase and uroporphyrinogen decarboxylase (FIG. 6) (Ponka P. 1997, Blood. 89, 1-25). In HBMS, daily ingestion of at least 350 mg. of iron reroutes the path of heme synthesis such that normal amounts of UROGEN I, and hence its product, URO I, are produced in a timely fashion (Rooney R. N. et al. unpublished data), which halts the disease progression.

In normal hepatic heme synthesis, porphyrin precursors are always produced; hence the associated neurotoxicity is also always present. URO I, the molecule that is the antidote to the neuron destruction associated with the porphyrin precursors, is also produced (FIG. 1). URO I cannot effectively counteract the neurotoxicity associated with the porphyrin precursors in the CNS unless it can enter into and circulate within the CNS. If it cannot, the CNS will sustain further damage in almost every instance of hepatic heme synthesis just as it does in the acute porphyrias.

Guamanian ALS has causes and effects (Banack S A et al. 2003 Neurology. 12;61(3), :387-9) (Ikemoto A et al 2000, Amyotroph Lateral Scler Other Motor Neuron Disord. March;1(2):97-104) similar to those of the acquired acute porphyria, porphyria turcica (Cripps D J. et al 1984, Br J Dermatol. 111(4), :413-22) that they may actually be the same disease though the toxic precipitator of Guamanian ALS, cycad nuts, was probably even more pernicious than the fungicides that precipitated porphyria turcica. Both were caused by ingested toxins that had contaminated the food supply and both, therefore, greatly increased demand for hepatic heme synthesis. Guamanian ALS was defined in 1952 (Donald Koerner and Jose Torres. 1952, Annals of Internal Medicine) and predated the Hoesch or Watson-Schwarz tests which quantify the porphyrin precursor, porphobilinogen (PBG), and are the still current and best tests for the acute neurotoxic porphyrias (Mauzerall D, Granick S., J Biol Chem 1956;219:435-446). Guamanian amyotrophic lateral sclerosis, therefore, could not have been accurately diagnosed as the acquired acute porphyria that it probably actually was.

The damage done by untreated HBMS is limited to damage to the motor cortex and to the sensory cortex. It is caused by a lack of the iron needed to support the production of normal amounts of URO I. ALS is most likely the result of the CNS becoming at least partially impermeable to URO I or to an inability to distribute URO I as normal within the CNS. Otherwise, the effects on the CNS are very closely matched though the etiology and parts of the pathogenesis are different. Although HBMS is inherited via a pattern of autosomal dominance with variable penetrance and is not sex linked, males are less likely to suffer neurological damage because they do not have the monthly iron loss associated with menstruation.

Relationship to Amyotrophic Lateral Sclerosis:

The rates of CNS destruction between the two “siblings”, ALS and HBMS, should be different because there are times when even untreated HBMS sufferers are able to produce sufficient URO I due to normal fluctuations in iron intake and availability. During those times, production and delivery of URO I is normal and no further CNS damage is incurred.

The second difference is that ALS does not include sensory deficit while HBMS does. Though future research will be required to precisely define this difference, it can be explained as follows: Untreated HBMS includes periods when production of the neurotoxicity associated porphyrin precursors is high while URO I production is low. CNS damage is inevitable during those periods.

In ALS, except for Guamanian ALS, there is no disturbance in hepatic heme synthesis so normal amounts of the porphyrin precursors are produced as is URO I and all enter the bloodstream in a time interval that is effective for preserving the CNS.

A plausible explanation of why ALS affects only the motor neurons while HBMS affects both sensory and motor neurons is that the ALS brain may remain partially permeable to URO I or mutated superoxide dismutase-1 (SOD1) structures may incompletely absorb URO I, allowing enough URO I into the CNS to protect the sensory cortex but not enough to continue on and protect the motor cortex as well.

This concept is supported by the very existence of Guamanian ALS because it is an example of the neurotoxicity associated porphyrin precursor production outpacing URO I production. That imbalance would explain why the effects of Guamanian ALS on the CNS would be identical to that of familial amyotrophic lateral sclerosis (FALS) and sporadic ALS even though their etiologies and probably their pathogeneses are different. URO I delivery would be timely but amounts would be insufficient. This echoes the pattern of the CNS damage of AIP.

URO I, if normal, has a small enough molecular size (FIG. 2) to penetrate the normal BBB while its isomer, URO III (FIG. 3), which is the product of uroporphyrinogen III, which is on the direct path to heme, is a larger molecule that cannot.

There is no record of a deficit of URO I or a surfeit of the porphyrin precursors in the serum or urine of ALS patients though that was necessarily true for Guamanian ALS and evidently went undetected, unrecorded or was ignored. There has also been no evidence that the URO I molecule produced in all cases of ALS has the normal structure (FIG. 2) that is best suited to penetrating the blood-brain barrier. There has been evidence of structures in the ALS brain that evidently either make the BBB less permeable to URO I or prevent its circulation to the motor neurons. The neurotoxicity associated with the porphyrin precursors is not blocked by the BBB, as is evidenced by the acute porphyrias. This may be due to the fact that a single molecule of porphobilinogen is even smaller than either of the two uroporphyrin isomers. (FIG. 5 as compared to FIG. 2 and FIG. 3).

SOD1 (Kato, S. et al. 2001, Neuropathology. 21(1): 67-81) found in familial ALS (FALS), is likely to be a cause of the blockage of the entry of URO I into the CNS or of inhibiting its distribution to the motor neurons by the neuronal Lewy body-like hyaline inclusions (LBHI) and astrocytic hyaline inclusions (Ast-HI) that are the hallmarks of FALS. The logic is that these products of the mutated SOD1 either clog the BBB such that entry of URO I into the CNS is either inhibited or halted altogether or that the structures produced by the SOD1 mutation absorb URO I preventing it from circulating as normal within the brain.

PCT, the most common of the porphyrias, is the only hepatic porphyria that is never neurotoxic and is the only hepatic porphyria characterized by excessive production of URO I. It is not known if Stephen Hawking, the most prominent example of a patient with slowly progressing ALS, has concomitant ALS and PCT.

There has been at least one report of an acute porphyria concomitant with PCT in which no nervous system damage was reported. (Doss M O et al. 2002, Med Klin (Munich) 97(1): 1-5).

There has also been a report of an iron porphyrin (FeTCPP) being helpful in slowing disease progression in a mouse of ALSAS et al. 2003, J Neurochem 85(1):142-50) This may be related to the feedback inhibition of the neurotoxicity associated porphyrin precursors, ALA and porphobilinogen (PBG) and may therefore not be safely sustainable on a long term basis just as hematin is not.

Other Disorders Capable of Stimulating Hepatic Heme Synthesis:

Most illnesses, injuries, infections, disorders or toxins of endogenous or exogenous origin stimulate hepatic heme synthesis, meaning that any that also involve the PNS and/or the CNS can be exacerbated by any of those triggers unless they are accompanied by commensurate increases in either or both of the uroporphyrin isomers, uroporphyrins I and/or III, meaning that CNS disorders that can be ameliorated by URO I include, but are not limited to, ALS, stroke, encephalitis, meningitis, and hereditary biochemical multiple sclerosis (HBMS). PNS disorders that can be ameliorated by URO III include, but are not limited to, AIDS related neuropathy, Guillaine-Barre syndrome and diabetic neuropathy.

### SUMMARY OF INVENTION

The present invention teaches a hitherto unknown value, purpose and use for naturally produced molecules that had hitherto been considered valueless. They are neuroprotectors, which can be used to halt, mitigate, prevent or preclude the neurological damage associated with other naturally produced molecules, the porphyrin precursors, ALA and PBG, that are also part of the hepatic heme biosynthetic pathway. The neuroprotectors are useful in halting or mitigating the neuron damage in some common neurological diseases, disorders and injuries. This invention gives rise to the objectives described below.

An objective of the invention is to use URO I to halt or mitigate the CNS damage that would otherwise occur as a result of the neurotoxicity associated with the presence of the porphyrin precursors, ALA and/or PBG.

Another objective of the invention is to use URO III to halt or mitigate the PNS damage that would otherwise occur as a result of the neurotoxicity associated with the presence of the porphyrin precursors, ALA and/or PBG.

### BRIEF DESCRIPTION OF DRAWINGS

The accompanying drawings illustrate the present invention. In these drawings:

FIG. 1 is a flow diagram that illustrates the position in heme synthesis normally occupied by UROGEN I.

FIG. 2 is a diagram of the molecular structure of URO I.

FIG. 3 is a diagram of the molecular structure of URO III.

FIG. 4 is a diagram of the molecular structure of ALA.

FIG. 5 is a diagram of the molecular structure of PBG.

FIG. 6 is a flow diagram of heme synthesis in untreated HBMS.

### DETAILED DESCRIPTION

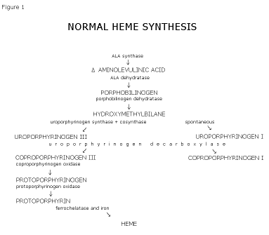

Referring now to FIG. 1, there is shown a flow diagram of normal heme synthesis. The heme biosynthetic pathway is a multistage process starting with glycin and succinyl-CoA and facilitated by several enzymes/catalysts that result in the end product of heme. The part of heme synthesis relevant to this invention begins with aminolevulinic acid synthase and culminates with heme. There is a straight line progression from aminolevulinic acid synthase to heme except for a spontaneous conversion of hydroxymethylbilane (HMB) to UROGEN I, progenitor of URO I, when uroporphyrinogen III (UROGEN III) synthase and UROGEN III co-synthase, which are steps in the path to heme and the progenitors of URO III, do not convert all of the available HMB to uroporphyrinogen III (UROGEN III). (Sassa S. 1996, Blood Review, 10, 53-58).

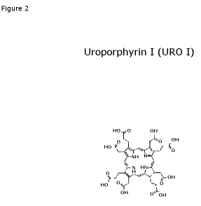

Referring now to FIG. 2, there is shown a diagram of the molecular structure of URO I. Normal URO I is the smaller of the two uroporphyrin isomer molecules and is therefore the one more capable of penetrating the BBB.

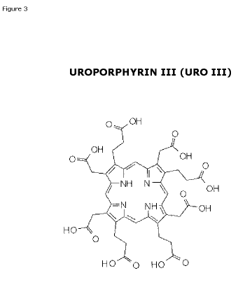

Referring now to FIG. 3, there is shown a diagram of the molecular structure of URO III. URO III is the larger of the two uroporphyrin isomer molecules and is therefore the one that is less capable of penetrating the BBB.

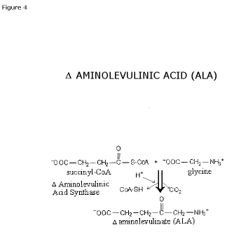

Referring now to FIG. 4, there is shown a diagram of the molecular structure of ALA. Aminolevulinic acid is one of the two porphyrin precursors that has been associated with neuron damage.

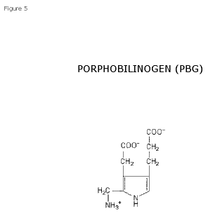

Referring now to FIG. 5, there is shown a diagram of the molecular structure of porphobilinogen (PBG). Porphobilinogen is the other porphyrin precursor that has been associated with neuron damage.

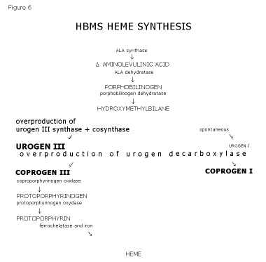

Referring now to FIG. 6, there is shown a diagram of the heme biosynthetic pathway in untreated HBMS. Comparisons to normal amounts are indicated by enlargement or reduction of type fonts as enzymes compare to enzymes, uroporphyrinogens (UROGENS) compare to UROGENS and coproporphyrinogens (COPROGENS) compare to COPROGENS. When there is insufficient iron availability in the liver, levels of uroporphyrinogen III synthase and cosynthase and of uroporphyrinogen decarboxylase are all overproduced. Overproduction of those enzymes/catalysts causes reductions in UROGEN I production and thereby causes increases of both of the coproporphyrinogen isomers, further depleting what little UROGEN I had been produced. This illustration is based upon the typical porphyrin counts of urinalyses during acute attack in a patient who had HBMS. This porphyrin distribution is based on several porphyrin urinalyses measured during exacerbations. (Rooney R N et al. unpublished data) That patient's disease is now arrested as a result of daily iron supplementation. (Rooney R N, et al. 1999, Am J Med Genet, 86(2), 194-196).

The above described drawings illustrate the background information necessary to understand the unique and previously unrecognized value of uroporphyrin(ogen) I (URO I), its position in the flow of heme synthesis, and its comparative molecular size in relation to the other pertinent molecules involved in hepatic heme synthesis.

The background information details why URO I, whose importance as a neuroprotector had not previously been recognized, is actually vital to healthy CNS function. Since that value has now been recognized thanks to the discovery of HBMS, it must be assured that URO I be made available to the CNS whenever the porphyrin precursors and their associated neurotoxicity are present.

URO I is currently readily available on the medical marketplace for use in laboratory experiments and has been used for years to piggyback antibodies that attach to cancer cells so that the photosensitive properties of porphyrins would then render the cancer cells vulnerable to destruction by light.

Implementation of this invention contemplates a medication that entails the purification of URO I and URO III to medical grade standards, determination of formulations suitable for the various possible modes of delivery, determination of correct dosages and Food and Drug Administration (FDA) approval for its use as a medication for humans.

This invention was made possible by the discovery of HBMS, which supplies the missing link that connects and gives new meaning to research done more than fifty years ago and on through research completed in 2003, less than fifty weeks ago.

### REFERENCES

Banack S A; Cox P A 2003, Biomagnification of cycad neurotoxins in flying foxes: Implications for ALS-PDC in Guam. Neurology 61(3):387-9.Cripps D J, Peters H A, Gocmen A. et al. 1984, Porphyria turcica due to hexachlorobenzene: a 20 to 30 year follow-up study on 204 patients. Br J Dermatol (England), 111(4) p413-22.

Donald Koerner and Jose Torres. 1952, Annals of Internal Medicine.

Doss M O, Gross U, Puy H, et al. 2002, [Coexistence of hereditary coproporphyria and porphyria cutanea tarda: a new form of dual porphyria] Med Klin (Munich) (Germany), 97(1) p1-5.

Hindmarsh J T, Oliveras L, Greenway D C. 1999, Biochemical differentiation of the porphyrias. Clin Biochem, 32(8):609-19.

Ikemoto A, Hirano A, Akiguchi I 2000, Neuropathology of amyotrophic lateral sclerosis with extra-motor system degeneration: characteristics and differences in the molecular pathology between ALS with dementia and Guamanian ALS. Amyotroph Lateral Scler Other Motor Neuron Disord. 1(2):97-104.

Kato S, Nakashima K, Horiuchi S, et al. 2001, Formation of advanced glycation end-product-modified superoxide dismutase-1 (SOD1) is one of the mechanisms responsible for inclusions common to familial amyotrophic lateral sclerosis patients with SOD1 gene mutation, and transgenic mice expressing human SOD1 gene mutation. Neuropathology. 21(1): 67-81.

Khanderia U. 1986, Circulatory collapse associated with hemin therapy for acute intermittent porphyria. Clin Pharm 5(8):690-2.

Kotze M J, de Villiers J N P, Rooney R N, Grobbelaar J J, Mansvelt E P G, Bouwens C S H, Carr J, Stander I, du Plessis L. Analysis of the NRAMP1 gene implicated in iron transport: association with multiple sclerosis and age effects: Blood Cells, Molecules, and Diseases 2001; 27: 44-53.

Kuhnel A, Gross U, Doss M O. 2000, Hereditary coproporphyria in Germany: clinical-biochemical studies in 53 patients. Clin Biochem August;33(6):465-73.

Lip G Y, McColl K E, Moore M R I. 1993, The acute porphyrias. Br J Clin Pract 47(1):38-43.

Lundin G, Lee J S, Thunell S, et al. 1997, Genetic investigation of the porphobilinogen deaminase gene in Swedish acute intermittent porphyria families. Hum Genet 100(1):63-6.

Nishioka E, Funasaka Y, Bito T, et al. 2003, Porphyria cutanea tarda with menopausal exacerbation: the possible role of menstruation as natural phlebotomy. J Am Acad Dermatol, 49(3):547-50.

Nordmann Y; Puy H. 2002, Human hereditary hepatic porphyrias. Clin Chim Acta, 325(1-2):17-37).

Ong P M, Lanyon W G, Hift R J, et al. 1998, Identification of two novel mutations in the hydroxymethylbilane synthase gene in three patients from two unrelated families with acute intermittent porphyria. Hum Hered 48(1):24-9.

Mauzerall D, Granick S. 1956, J Biol Chem 219:435-446.

Rooney R N, Kotze M J, de Villiers J N P, Hillermann R, Cohen J A. 1999, Multiple sclerosis, porphyria-like symptoms, and a history of iron deficiency anemia in a family of Scottish descent. Am J Med Genet, 86(2), 194-196.

Wu A S; Kiaei M; Aguirre N; Crow J P; Calingasan N Y; Browne S E; Beal M F. 2003, Iron porphyrin treatment extends survival in a transgenic animal model of amyotrophic lateral sclerosis. J Neurochem, 85(1):142-50.

Parameters:

Since other modifications and changes varied to fit particular operating requirements and environments will be apparent to those skilled in the art, the invention is not limited to the examples chosen for purposes of disclosure, and covers all changes and modifications which do not constitute departures from the true spirit and scope of this invention.

Having thus described the invention, what is desired to be protected by Letters Patent is presented in the subsequently presented claims.

1. A method of treatment for common neurological disorders of the central and peripheral nervous systems wherein the neuron damage associated with the porphyrin precursors, delta-aminolevulinic acid and/or porphobilinogen, is a contributing factor, comprising the use of a uroporphyrin isomer as a neuroprotector to halt or mitigate the neuron damage.

2. The method of claim 1 wherein the neurological disorder is selected from the group of disorders: amyotrophic lateral sclerosis, stroke, encephalitis, meningitis, hereditary biochemical multiple sclerosis, acute immunodeficiency syndrome related neuropathy, diabetic neuropathy, Guillaine-Barre syndrome. [claim Reference]

3. The method of claim 2 (amyotrophic lateral sclerosis) further comprising the use of uroporphyrin I delivered to the central nervous system using any of several methods that penetrate or bypass the blood-brain barrier. [claim Reference]

4. The method of claim 3 further comprising the use of intrathecal delivery, intravenous injection, infusion, ingestion, olfactory (nasal) delivery, delivery by shunt or stent directly into the brain, or BBB weakening drugs or other methods meant to penetrate or bypass the blood-brain barrier to improve uroporphyrin I entry into or distribution throughout the central nervous system. [claim Reference]

5. The method of claim 1 wherein there is a neurological disorder of the central nervous system (stroke, encephalitis, meningitis). [claim Reference]

6. The method of claim 5 further comprising the use of uroporphyrin I delivered to the central nervous system using any of several methods. [claim Reference]

7. The method of claim 6 further comprising the use of intrathecal delivery, intravenous injection, infusion, ingestion, olfactory (nasal) delivery, delivery by shunt or stent directly into the brain, or BBB weakening drugs or other methods meant to penetrate or bypass the blood-brain barrier to improve uroporphyrin I entry into or distribution throughout the central nervous system. [claim Reference]

8. The method of claim 1 wherein the neurological disorder is hereditary biochemical multiple sclerosis [claim Reference].

9. The method of claim 8 further comprising the use of uroporphyrin I delivered to the central nervous system during acute atacks using any of several methods. [claim Reference]

10. The method of claim 9 further comprising the use of intrathecal delivery, intravenous injection, infusion, ingestion, olfactory (nasal) delivery, delivery by shunt or stent directly into the brain, or BBB weakening drugs or other methods meant to penetrate or bypass the blood-brain barrier to improve uroporphyrin I entry into or distribution throughout the central nervous system. [claim Reference]

11. The method of claim 1 wherein the neurological disorder is a peripheral nervous system disorder (acute immunodeficiency syndrome related neuropathy, diabetic neuropathy, Guillaine-Barre syndrome). [claim Reference]

12. The method of claim 11 further comprising the delivery of higher than normal amounts of uroporphyrin III to the bloodstream. [claim Reference]

13. The method of claim 12 further comprising the use of intravenous injection, infusion, osmotic absorption or ingestion to deliver the uroporphyrin III to the bloodstream. [claim Reference]

14. The method according to claim 1 wherein aminolevulinic acid dehydratase and/or porphobilinogen dehydratase are increased in serum or the central nervous system for the purpose of synthesizing uroporphyrin I anywhere within the body. [claim Reference]

15. The method of claim 1 wherein uroporphyrin I can be effectively substituted for or combined with uroporphyrin III though uroporphyrin III cannot be substituted for uroporphyrin I. [claim Reference]

16. The method of claim 1 wherein the treatment applies to, but is not limited to, the above listed neurological disorders. [claim Reference]
